(19) United States
(12) Patent Application Publication
(10) Pub. No.: US 2022/0162154 A1
(43) Pub. Date: May 26, 2022

(54) D-METYROSINE COMPOSITIONS AND METHODS FOR PREPARING SAME

(71) Applicant: TYME, INC, Bedminster, NJ (US)

(72) Inventor: John ZUCARO, Newark, DE (US)

(21) Appl. No.: 17/440,415

(22) PCT Filed: Mar. 18, 2020
(86) PCT No.: PCT/US2020/023299

§ 371 Date: Sep. 17, 2021

Related U.S. Application Data
(60) Provisional application No. 62/822,242, filed on Mar. 22, 2019.

(51) Int. Cl. C07C 227/18; B01J 21/18; B01J 23/44

(52) U.S. Cl. CPC C07C 227/18; B01J 23/44; B01J 21/18

(57) ABSTRACT

The disclosure provides processes for preparing a compound of formula I, comprising reacting a compound of formula II with an aqueous acid in a solvent and at a temperature sufficient for at least about 48 hours to produce a compound of formula I: wherein, R¹-R⁵ are defined herein. Also provided are D-metyrosine prepared according to the processes described herein and compositions comprising the D-metyrosine provided herein.

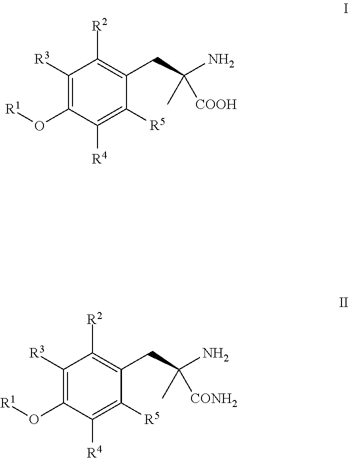

### CROSS-REFERENCED TO RELATED APPLICATIONS

This application claims priority to U.S. Provisional Patent Application No. 62/822,242, filed Mar. 22, 2019, the disclosure of which is incorporated by reference herein.

### TECHNICAL FIELD

This invention relates to D-metyrosine compositions and methods for their preparation.

### BACKGROUND

Metyrosine is an inhibitor of the enzyme tyrosine hydroxylase and depletes levels of the catecholamines, such as dopamine, adrenaline and noradrenaline, when administered to patients. L-metyrosine is useful in the treatment high blood pressure in patients having pheochromocytoma, an adrenal gland cancer.

What is needed are alternate techniques for preparing metyrosine.

### SUMMARY

In certain embodiments, the disclosure provides processes for preparing a compound of formula I, comprising reacting a compound of formula II with an aqueous acid in a solvent and at a temperature sufficient for at least about 48 hours to produce a compound of formula I:

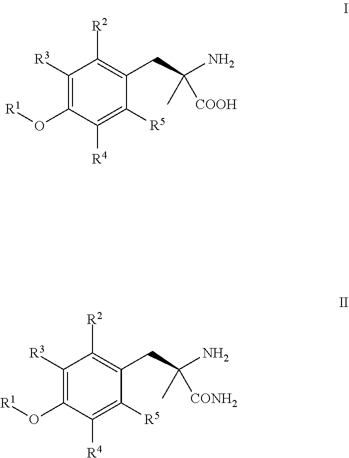

wherein, R¹-R⁵ are defined herein. In some aspects, the compound of formula I is D-metyrosine (compound 1). In other aspects, the compound of formula II is compound 2:

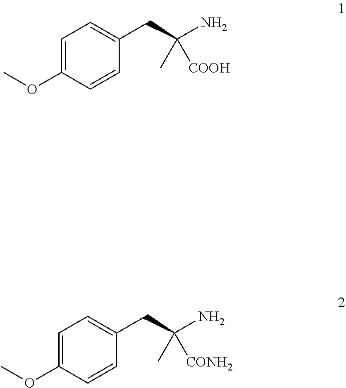

In other embodiments, the disclosure provides D-metyrosine prepared according to the processes described herein.

In further embodiments, the disclosure provides composition comprising the D-metyrosine provided herein. In some aspects, the compositions comprise a mixture of D-metyrosine and L-metyrosine. In further aspects, the compositions comprises a mixture that comprises at least about 50 wt %, based on the weight of the composition, of D-metyrosine.

In yet other embodiments, the disclosure provides a compound that is compound 2, compound 3, compound 4, compound 5, or compound 6:

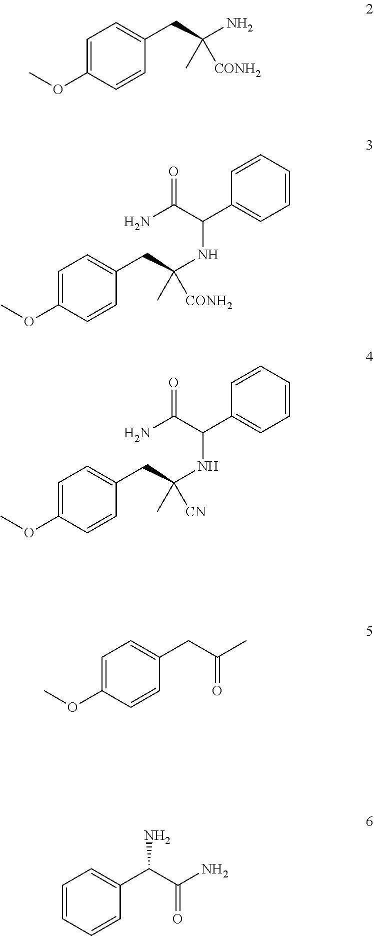 [SMILES: COc1ccc(C[C@@](C)(N)C(N)=O)cc1 ; COc1ccc(C[C@@](C)(NC(C(N)=O)c2ccccc2)C(N)=O)cc1 ; COc1ccc(C[C@](C)(C#N)NC(C(N)=O)c2ccccc2)cc1 ; COc1ccc(CC(C)=O)cc1 ; NC(=O)[C@@H](N)c1ccccc1]

or a salt or solvate thereof.

Other aspects and embodiments of the invention will be readily apparent from the following detailed description of the invention.

### DETAILED DESCRIPTION OF THE DRAWINGS

The present application is further understood when read in conjunction with the appended drawings. For the purpose of illustrating the subject matter, there are shown in the drawings exemplary embodiments of the subject matter; however, the presently disclosed subject matter is not limited to the specific compositions, methods, devices, and systems disclosed. In addition, the drawings are not necessarily drawn to scale.

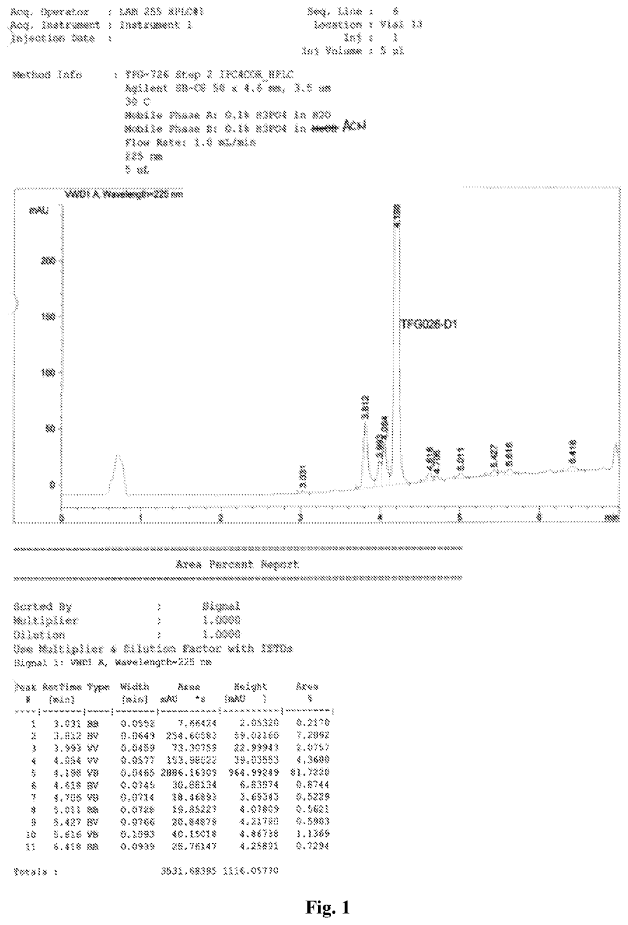

FIG. 1 is the high-performance liquid chromatogram (HPLC) for compound TFG026-D1 using an Agilent SB-C8 50×4.6 mm, 3.5 μm column at 30° C., using 0.1% H₃PO₄ in H₂O (mobile phase A), 0.1% H₃PO₄ in acetonitrile (mobile phase B), a flow rate of 1.0 mL/min, and 225 nm wavelength.

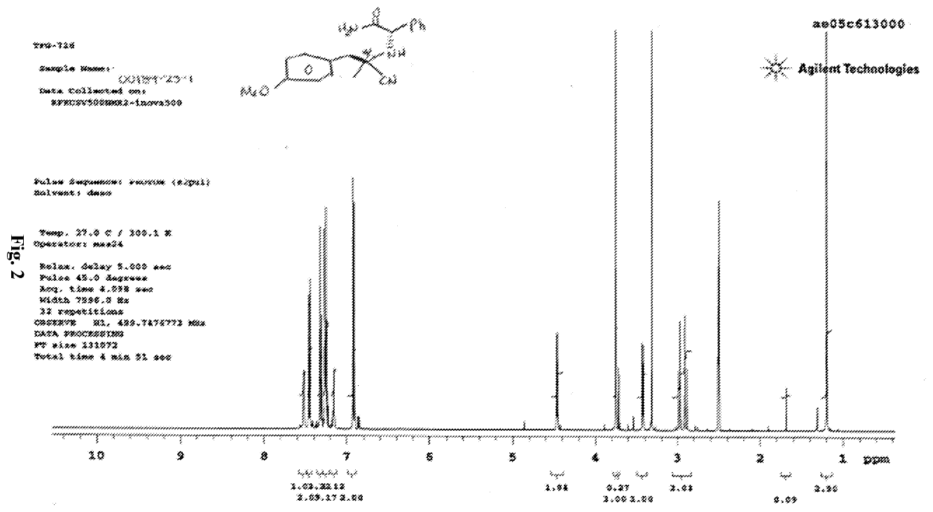

FIG. 2 is the proton nuclear magnetic resonance (¹H-NMR) spectrum (500 MHz) for compound TFG026-D1 in DMSO.

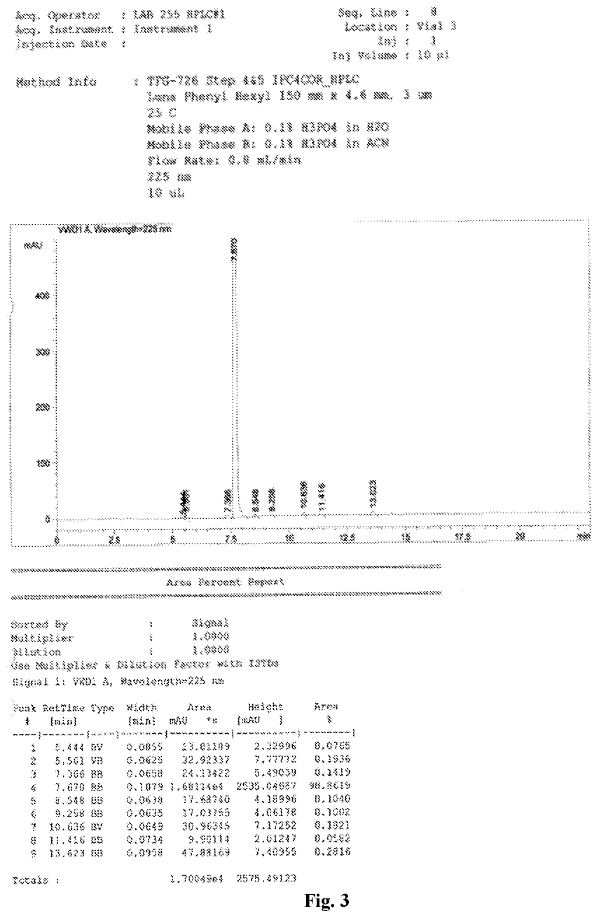

FIG. 3 is the HPLC chromatograph for compound TFG026-D2 using a Luna phenyl hexyl 150×4.6 mm, 3 μm chromatograph, 25° C., 0.1% H₃PO₄ in H₂O (mobile phase A), 0.1% H₃PO₄ in acetonitrile (mobile phase B), flow rate of 0.8 mL/min, and 225 nm wavelength.

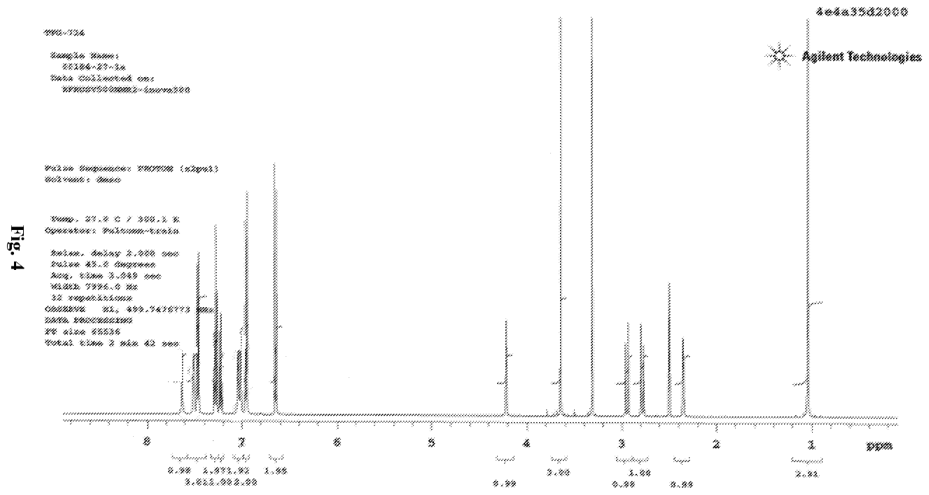

FIG. 4 is the ¹H-NMR spectrum (500 MHz) for compound TFG026-D2 in DMSO.

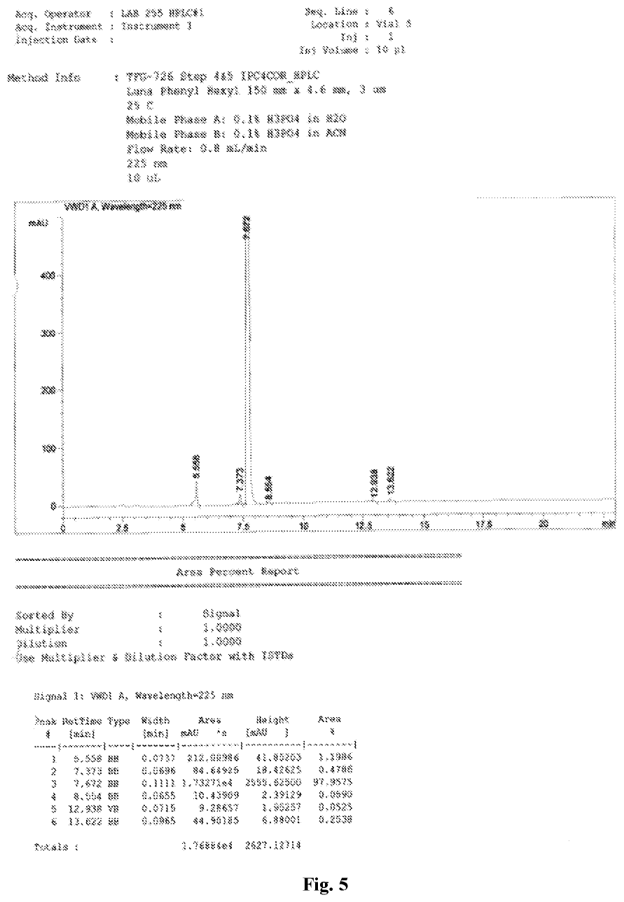

FIG. 5 is the HPLC chromatograph of compound TFG026-D2-pure using a Luna phenyl hexyl 150×4.6 mm, 3 μm chromatograph, 25° C., 0.1% H₃PO₄ in H₂O (mobile phase A), 0.1% H₃PO₄ in acetonitrile (mobile phase B), flow rate of 0.8 mL/min, and 225 nm wavelength.

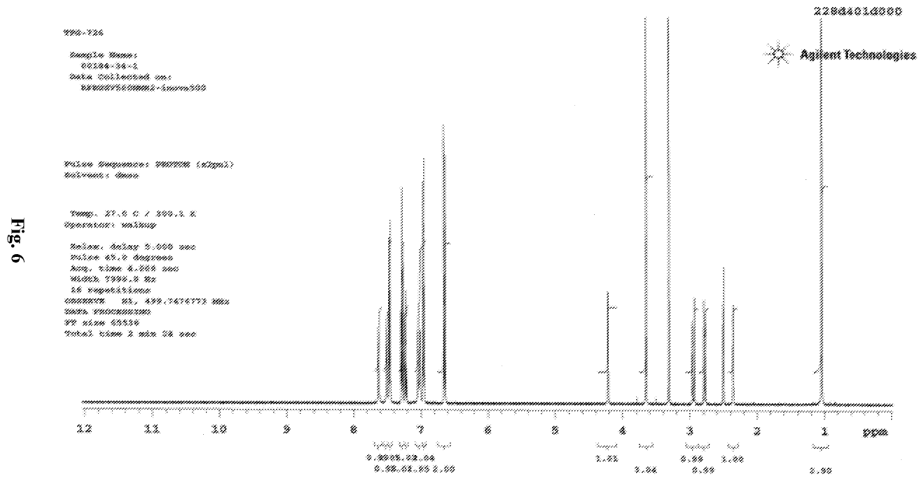

FIG. 6 is the ¹H-NMR spectrum (500 MHz) of compound TFG026-D2-pure in DMSO.

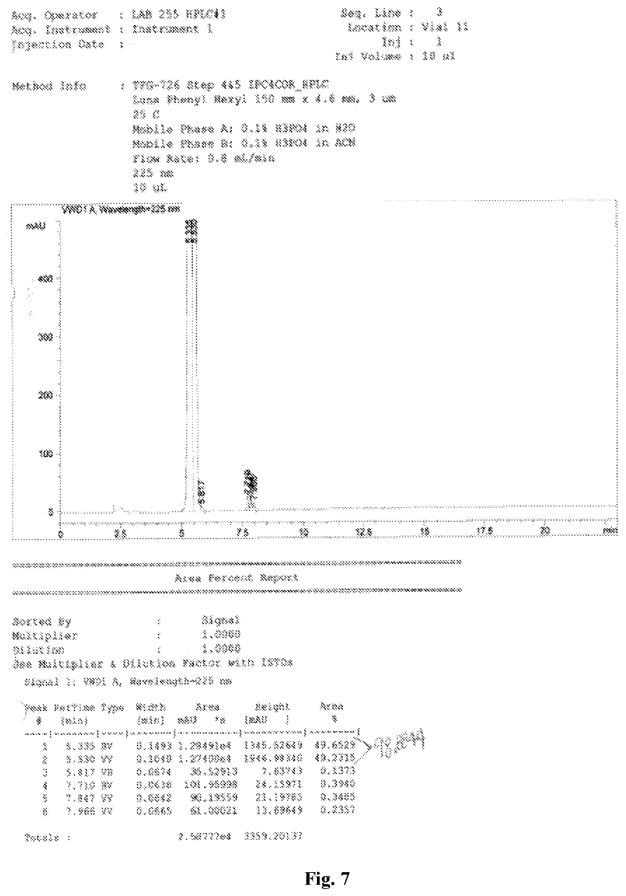

FIG. 7 is the HPLC chromatograph of compound TFG026-D3 using a Luna phenyl hexyl 150×4.6 mm, 3 μm chromatograph, 25° C., 0.1% H₃PO₄ in H₂O (mobile phase A), 0.1% H₃PO₄ in acetonitrile (mobile phase B), flow rate of 0.8 mL/min, and 225 nm wavelength.

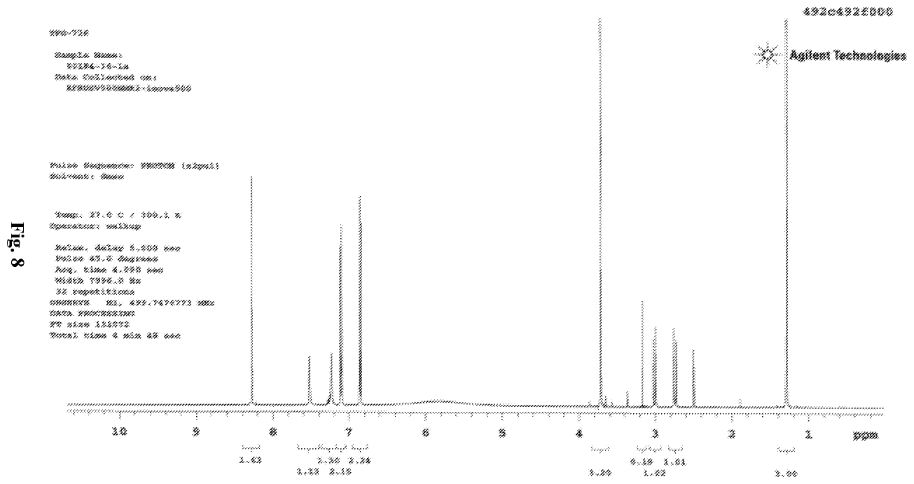

FIG. 8 is the ¹H-NMR spectrum (500 MHz) of compound TFG026-D3 in DMSO.

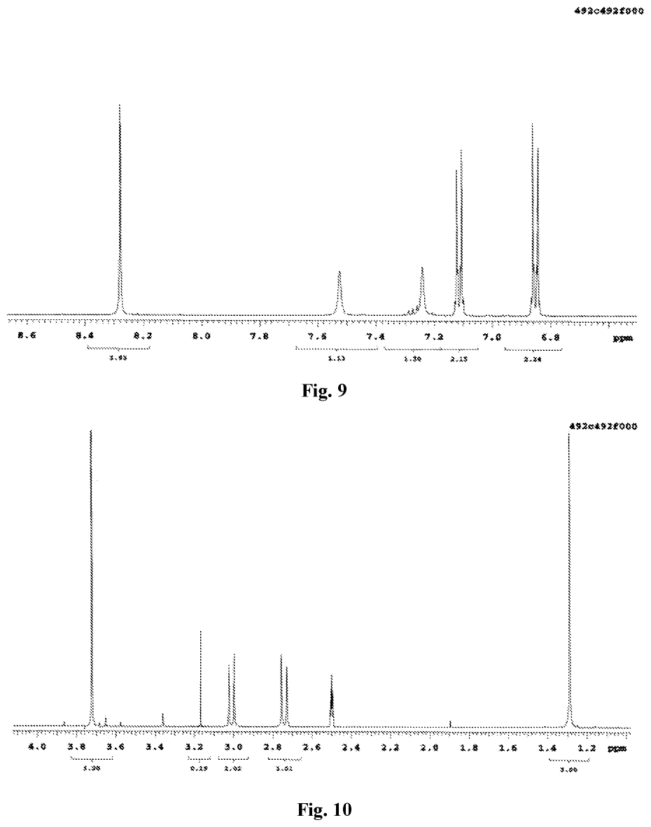

FIG. 9 is the ¹H-NMR spectrum downfield subset of FIG. 8.

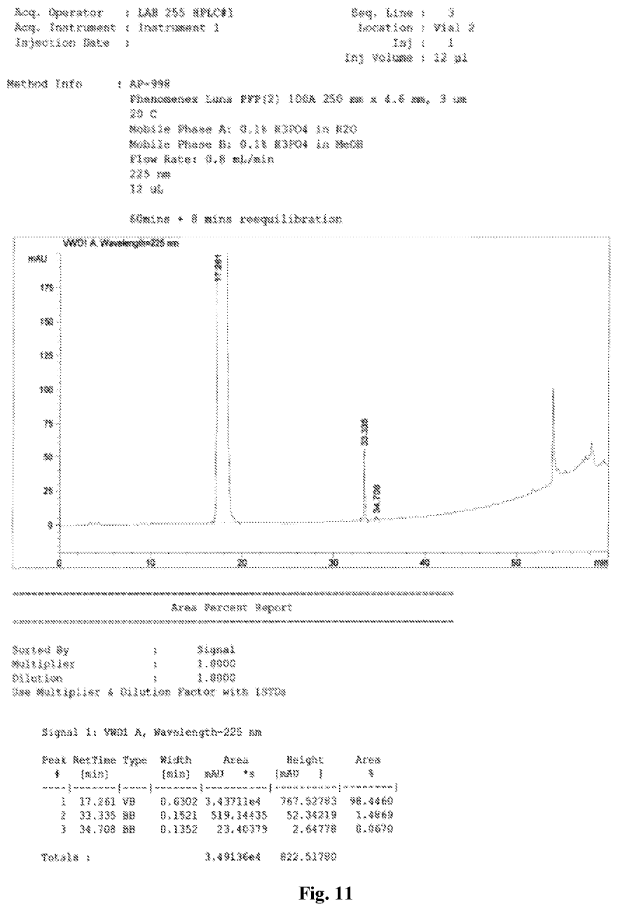

FIG. 10 is the ¹H-NMR spectrum upfield subset of FIG. 8.

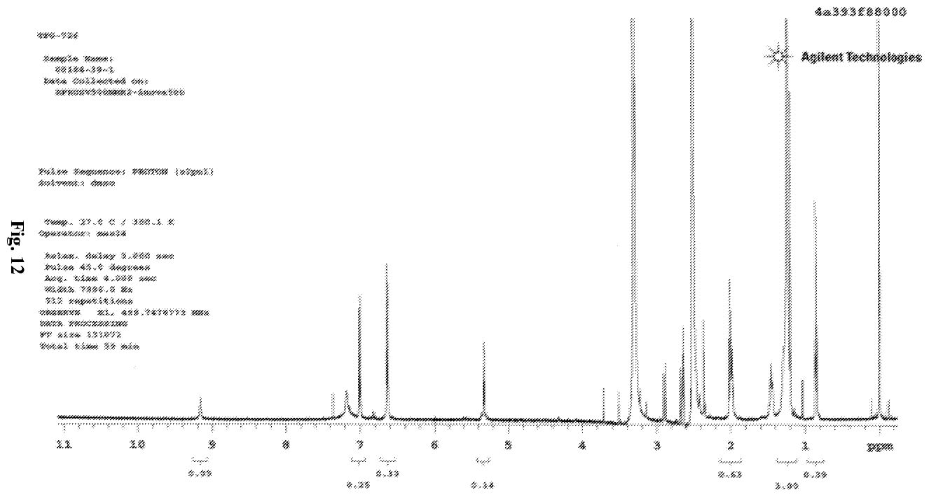

FIG. 11 is the HPLC chromatograph of compound TFG026-D4 using a Phenomenex Luna PFP (2) 100 A 250×4.6 mm, 3 μm chromatograph, 20° C., 0.1% H₃PO₄ in H₂O (mobile phase A), 0.1% H₃PO₄ in acetonitrile (mobile phase B), flow rate of 0.8 mL/min, and 225 nm wavelength.

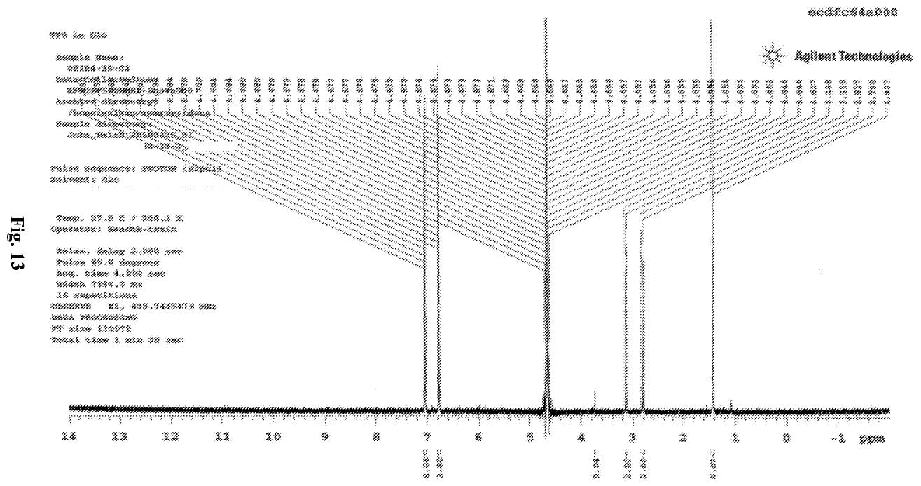

FIG. 12 is the ¹H-NMR spectrum (500 MHz) of compound TFG026-D4 in DMSO.

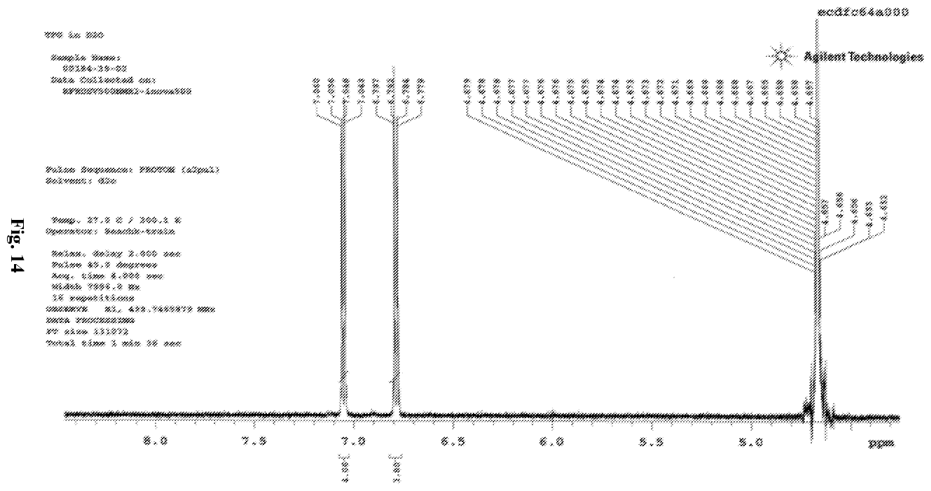

FIG. 13 is the ¹H-NMR spectrum (500 MHz) of compound TFG026-D4 in D₂O.

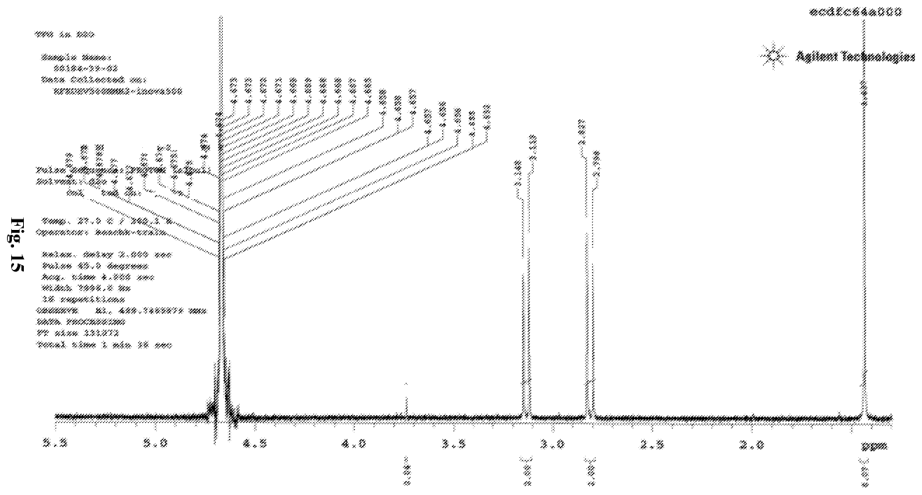

FIG. 14 is the ¹H-NMR spectrum downfield subset of FIG. 13.

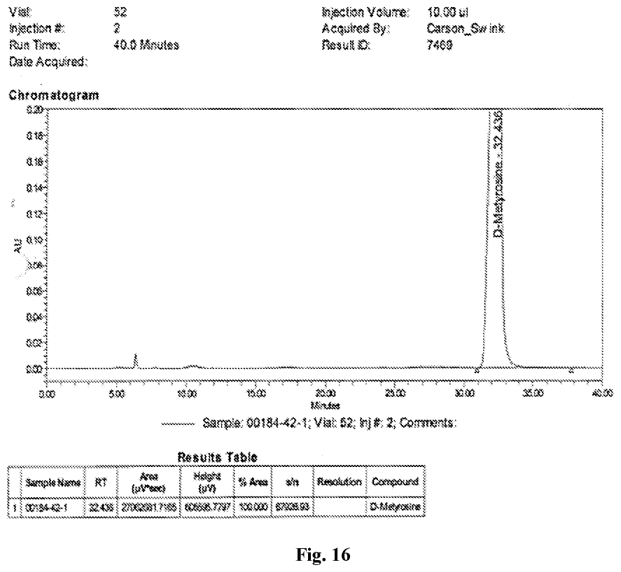

FIG. 15 is the ¹H-NMR spectrum midfield subset of FIG. 13.

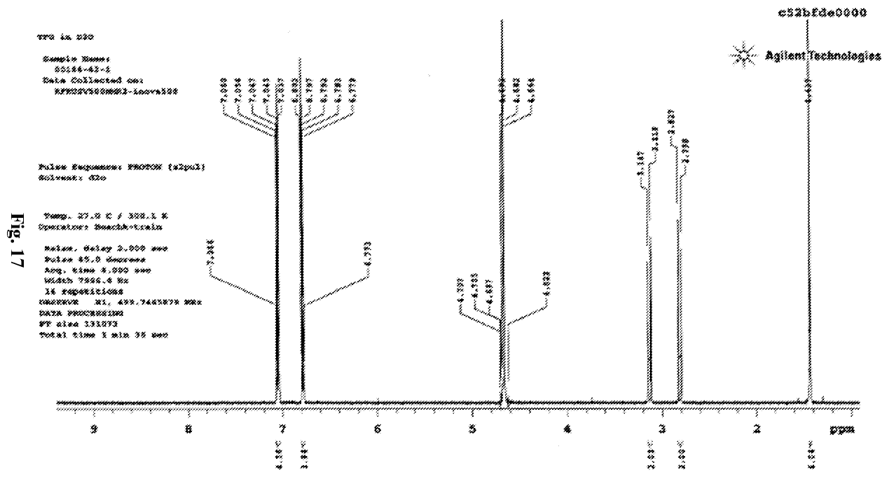

FIG. 16 is the HPLC chromatogram of compound TGF026-D4-pure.

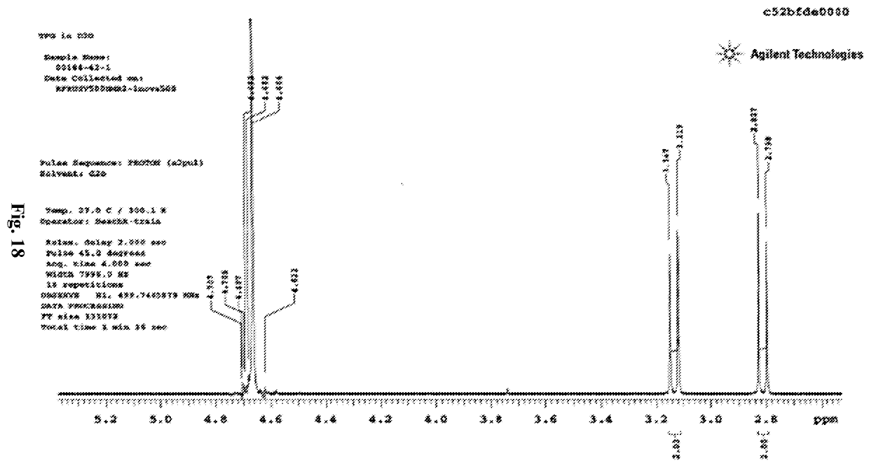

FIG. 17 is the ¹H-NMR spectrum (500 MHz) of compound TGF026-D4-pure in D₂O.

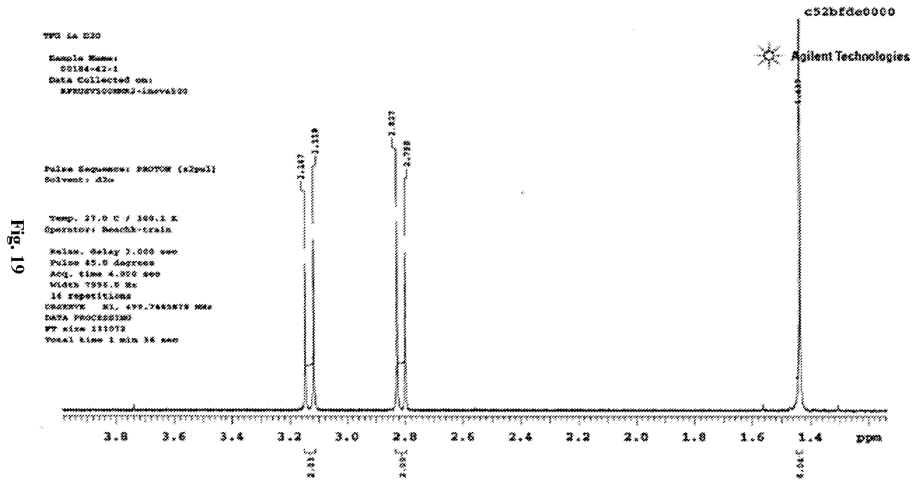

FIG. 18 is the ¹H-NMR spectrum midfield subset of FIG. 17.

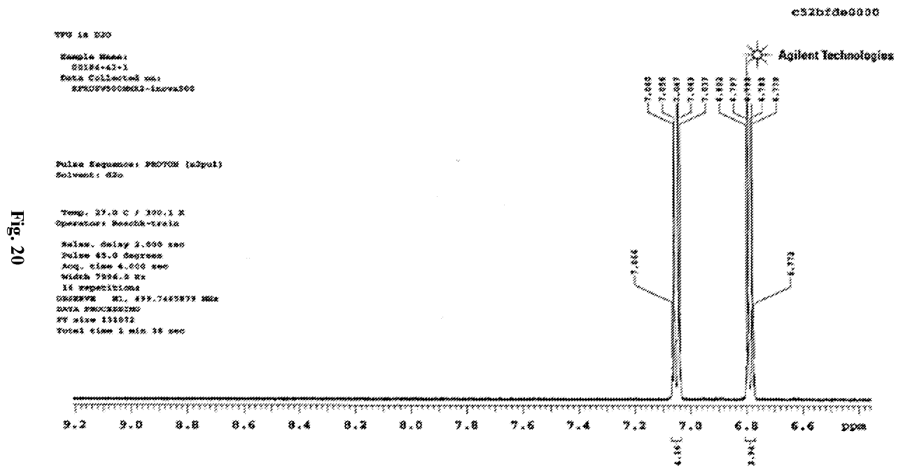

FIG. 19 is the ¹H-NMR spectrum upfield subset of FIG. 17.

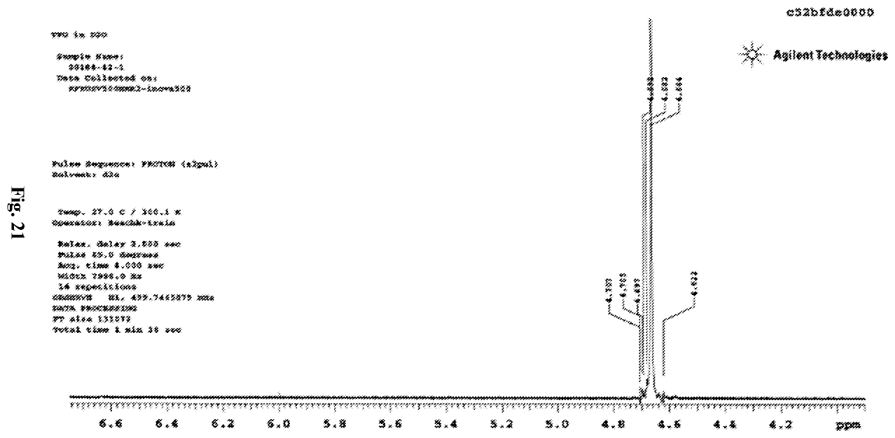

FIG. 20 is the ¹H-NMR spectrum upfield subset #1 of FIG. 19.

FIG. 21 is the ¹H-NMR spectrum upfield subset #2 of FIG. 19.

### DETAILED DESCRIPTION OF ILLUSTRATIVE EMBODIMENTS

In the present disclosure the singular forms “a”, “an” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When a value is expressed as an approximation by use of the descriptor “about” it will be understood that the particular value forms another embodiment. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about”. In other cases, the gradations used in a series of values may be used to determine the intended range available to the term “about” for each value. Where present, all ranges are inclusive and combinable. That is, references to values stated in ranges include every value within that range.

The term “alkyl” refers to an aliphatic group having 1 to 6 carbon atoms, e.g., 1, 2, 3, 4, 5, or 6 carbon atoms and includes, for example, methyl, ethyl, propyl, butyl, pentyl, or hexyl. An alkyl may be optionally substituted with one, two, or three substituents selected from halo (F, Cl, Br, or I, preferably F), —OH, —OC₁₋₆alkyl, —CN, —NH₂, —NH(C₁₋₆alkyl), or —NH(C₁₋₆alkyl)₂.

The term “alkoxy” refers to an —O-alkyl, with alkyl defined above. An alkoxy may be optionally substituted with one, two, or three substituents selected from halo (F, Cl, Br, or I, preferably F), —OH, —OC₁₋₆alkyl, —CN, —NH₂, —NH(C₁₋₆alkyl), or —NH(C₁₋₆alkyl)₂.

The term “cycloalkyl” refers to a cyclic aliphatic having 3 to 8 carbon atoms, e.g., 3, 4, 5, 6, 7, or 8 carbon atoms and includes, for example, cyclopropyl, cyclobutyl, cyclopentyl, cyclohexyl, cycloheptyl, or cyclooctyl. A cycloalkyl may be optionally substituted with one, two, or three substituents selected from halo (F, Cl, Br, or I, preferably F), —OH, —OC₁₋₆alkyl, —CN, —NH₂, —NH(C₁₋₆alkyl), or —NH(C₁₋₆alkyl)₂.

The term “alkenyl” refers to an aliphatic group having 2 to 6 carbon atoms, e.g., 2, 3, 4, 5, or 6 carbon atoms and at least one point of unsaturation that is a double bond. Thus, alkenyl includes, for example, ethenyl, propenyl, butenyl, pentenyl, or hexenyl. An alkenyl may be optionally substituted with one, two, or three substituents selected from halo (F, Cl, Br, or I, preferably F), —OH, —C₁₋₆alkyl, —OC₁₋₆alkyl, —CN, —NH₂, —NH(C₁₋₆alkyl), or —NH(C₁₋₆alkyl)₂.

The term “halogen” or “halo” as used herein refers to CI, Br, F, or I groups.

The term “aryl” refers to 6-15 membered monoradical bicyclic or tricyclic hydrocarbon ring systems, including bridged, spiro, and/or fused ring systems, in which at least one of the rings is aromatic. An aryl group may contain 6 (i.e., phenyl) or about 9 to about 15 ring atoms, such as 6 (i.e., phenyl) or about 9 to about 11 ring atoms. In certain embodiments, aryl groups include, but are not limited to, naphthyl, indanyl, indenyl, anthryl, phenanthryl, fluorenyl, 1,2,3,4-tetrahydronaphthalenyl, 6,7,8,9-tetrahydro-5H-benzocycloheptenyl, and 6,7,8,9-tetrahydro-5H-benzocycloheptenyl. In some embodiments, the aryl is naphthyl. An aryl may be optionally substituted with one, two, or three substituents selected from halo (F, Cl, Br, or I, preferably F), —OH, —OC₁₋₆alkyl, —CN, —NH₂, —NH(C₁₋₆alkyl), or —NH(C₁₋₆alkyl)₂.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments, “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself.

In view of the advantages provided by D-metyrosine compositions, processes for their preparation are provided. The terms “D-metyrosine,” “D-α-metyrosine,” and “D-α-methyl-tyrosine” as used herein are interchangeably and refer to 2-amino-3-(4-hydroxyphenyl)-2-methylpropanoic acid. D-metyrosine has the following structure:

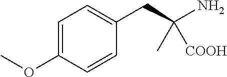

Thus, the present disclosure provided processes for preparing compound of formula I.

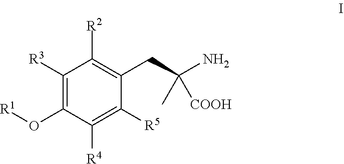

In these compounds, R¹ is C₁₋₆alkyl, C₃₋₈cycloalkyl, or aryl. In some embodiments, R¹ is C₁₋₆alkyl, such as methyl, ethyl, propyl, butyl, pentyl, or hexyl. Preferably, R¹ is methyl. In other embodiments, R¹ is C₃₋₈ cycloalkyl, such as cyclopropyl, cyclobutyl, cyclopentyl, cyclohexyl, cycloheptyl, or cyclooctyl. In further embodiments, R¹ is aryl, such as phenyl.

R² to R⁵ are, independently, H, halo, C₁₋₆alkyl, C₁₋₆alkoxy, C₃₋₈cycloalkyl, or aryl. In some embodiments, R² is H, halo, C₁₋₆alkyl, C₁₋₆alkoxy, C₃₋₈cycloalkyl, or aryl. In other embodiments, R² is H. In further embodiments, R² is halo such as F, Cl, Br, or I. In still other embodiments, R² is C₁₋₆alkyl such as methyl, ethyl, propyl, butyl, pentyl, or hexyl. In yet further embodiments, R² is C₁₋₆alkoxy, such as methoxy, ethoxy, propoxy, butoxy, pentoxy, or hexoxy. In other embodiments, R² is C₃₋₈cycloalkyl, such as cyclopropyl, cyclobutyl, cyclopentyl, cyclohexyl, cycloheptyl, or cyclooctyl. In further embodiments, R² is aryl, such as phenyl. Preferably, R² is H. In some embodiments, R³ is H, halo, C₁₋₆alkyl, C₁₋₆alkoxy, C₃₋₈cycloalkyl, or aryl. In other embodiments, R³ is H. In further embodiments, R³ is halo such as F, Cl, Br, or I. In still other embodiments, R³ is C₁₋₆alkyl such as methyl, ethyl, propyl, butyl, pentyl, or hexyl. In yet further embodiments, R³ is C₁₋₆alkoxy, such as methoxy, ethoxy, propoxy, butoxy, pentoxy, or hexoxy. In other embodiments, R³ is C₃₋₈cycloalkyl, such as cyclopropyl, cyclobutyl, cyclopentyl, cyclohexyl, cycloheptyl, or cyclooctyl. In further embodiments, R³ is aryl, such as phenyl. Preferably, R³ is H. In some embodiments, R⁴ is H, halo, C₁₋₆alkyl, C₁₋₆alkoxy, C₃₋₈cycloalkyl, or aryl. In other embodiments, R⁴ is H. In further embodiments, R⁴ is halo such as F, Cl, Br, or I. In still other embodiments, R⁴ is C₁₋₆alkyl such as methyl, ethyl, propyl, butyl, pentyl, or hexyl. In yet further embodiments, R⁴ is C₁₋₆alkoxy, such as methoxy, ethoxy, propoxy, butoxy, pentoxy, or hexoxy. In other embodiments, R⁴ is C₃₋₈cycloalkyl, such as cyclopropyl, cyclobutyl, cyclopentyl, cyclohexyl, cycloheptyl, or cyclooctyl. In further embodiments, R⁴ is aryl, such as phenyl. Preferably, R⁴ is H. In some embodiments, R⁵ is H, halo, C₁₋₆alkyl, C₁₋₆alkoxy, C₃₋₈cycloalkyl, or aryl. In other embodiments, R⁵ is H. In further embodiments, R² is halo such as F, Cl, Br, or I. In still other embodiments, R⁵ is C₁₋₆alkyl such as methyl, ethyl, propyl, butyl, pentyl, or hexyl. In yet further embodiments, R⁵ is C₁₋₆alkoxy, such as methoxy, ethoxy, propoxy, butoxy, pentoxy, or hexoxy. In other embodiments, R⁵ is C₃₋₈cycloalkyl, such as cyclopropyl, cyclobutyl, cyclopentyl, cyclohexyl, cycloheptyl, or cyclooctyl. In further embodiments, R⁵ is aryl, such as phenyl. Preferably, R⁵ is H.

Preferably, all of R² to R⁵ are H. More preferably, the compound of formula I is D-metyrosine.

The methods for preparing the compounds of formula I include reacting a compound of formula II with an aqueous acid in a solvent and at a temperature sufficient for at least about 48 hours.

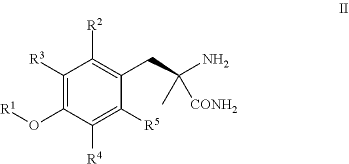

In these compounds of formula II, R¹ is C₁₋₆alkyl, C₃₋₈cycloalkyl, or aryl. In some embodiments, R¹ is C₁₋₆alkyl, such as methyl, ethyl, propyl, butyl, pentyl, or hexyl. Preferably, R¹ is methyl. In other embodiments, R¹ is C₃₋₈ cycloalkyl, such as cyclopropyl, cyclobutyl, cyclopentyl, cyclohexyl, cycloheptyl, or cyclooctyl. In further embodiments, R¹ is aryl, such as phenyl.

R² to R⁵ are, independently, H, halo, C₁₋₆alkyl, C₁₋₆alkoxy, C₃₋₈cycloalkyl, or aryl. In some embodiments, R² is H, halo, C₁₋₆alkyl, C₁₋₆alkoxy, C₃₋₈cycloalkyl, or aryl. In other embodiments, R² is H. In further embodiments, R² is halo such as F, Cl, Br, or I. In still other embodiments, R² is C₁₋₆alkyl such as methyl, ethyl, propyl, butyl, pentyl, or hexyl. In yet further embodiments, R² is C₁₋₆alkoxy, such as methoxy, ethoxy, propoxy, butoxy, pentoxy, or hexoxy. In other embodiments, R² is C₃₋₈cycloalkyl, such as cyclopropyl, cyclobutyl, cyclopentyl, cyclohexyl, cycloheptyl, or cyclooctyl. In further embodiments, R² is aryl, such as phenyl. Preferably, R² is H. In some embodiments, R³ is H, halo, C₁₋₆alkyl, C₁₋₆alkoxy, C₃₋₈cycloalkyl, or aryl. In other embodiments, R³ is H. In further embodiments, R³ is halo such as F, Cl, Br, or I. In still other embodiments, R³ is C₁₋₆alkyl such as methyl, ethyl, propyl, butyl, pentyl, or hexyl. In yet further embodiments, R³ is C₁₋₆alkoxy, such as methoxy, ethoxy, propoxy, butoxy, pentoxy, or hexoxy. In other embodiments, R³ is C₃₋₈cycloalkyl, such as cyclopropyl, cyclobutyl, cyclopentyl, cyclohexyl, cycloheptyl, or cyclooctyl. In further embodiments, R³ is aryl, such as phenyl. Preferably, R³ is H. In some embodiments, R⁴ is H, halo, C₁₋₆alkyl, C₁₋₆alkoxy, C₃₋₈cycloalkyl, or aryl. In other embodiments, R⁴ is H. In further embodiments, R⁴ is halo such as F, Cl, Br, or I. In still other embodiments, R⁴ is C₁₋₆alkyl such as methyl, ethyl, propyl, butyl, pentyl, or hexyl. In yet further embodiments, R⁴ is C₁₋₆alkoxy, such as methoxy, ethoxy, propoxy, butoxy, pentoxy, or hexoxy. In other embodiments, R⁴ is C₃₋₈cycloalkyl, such as cyclopropyl, cyclobutyl, cyclopentyl, cyclohexyl, cycloheptyl, or cyclooctyl. In further embodiments, R⁴ is aryl, such as phenyl. Preferably, R⁴ is H. In some embodiments, R⁵ is H, halo, C₁₋₆alkyl, C₁₋₆alkoxy, C₃₋₈cycloalkyl, or aryl. In other embodiments, R⁵ is H. In further embodiments, R² is halo such as F, Cl, Br, or I. In still other embodiments, R⁵ is C₁₋₆alkyl such as methyl, ethyl, propyl, butyl, pentyl, or hexyl. In yet further embodiments, R⁵ is C₁₋₆alkoxy, such as methoxy, ethoxy, propoxy, butoxy, pentoxy, or hexoxy. In other embodiments, R⁵ is C₃₋₈cycloalkyl, such as cyclopropyl, cyclobutyl, cyclopentyl, cyclohexyl, cycloheptyl, or cyclooctyl. In further embodiments, R⁵ is aryl, such as phenyl. Preferably, R⁵ is H.

Preferably, all of R² to R⁵ are H. More preferably, R¹ is methyl and all of R² to R⁵ are H, i.e., the compound of formula II is compound 2.

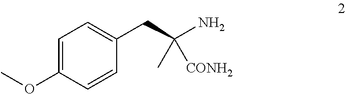

As described, the methods include reacting a compound of formula II with an aqueous acid in a solvent and at a temperature sufficient for at least about 48 hours. In some embodiments, the aqueous acid is an aqueous hydrogen halide, such as hydrogen chloride, hydrogen bromide, or hydrogen iodide. More preferably, the aqueous acid is hydrogen bromide. The solvent may be selected by one skill in the art from aqueous solvents. The term “aqueous” as used herein refers to a liquid containing at least about 10 vol %, based on the total volume of the liquid, of water. In some embodiments, an aqueous liquid contains at least about 20 vol %, about 30 vol %, about 40 vol %, about 50 vol %, about 60 vol %, about 70 vol %, about 80 vol %, about 90 vol %, about 95 vol %, or about 99 vol %, based on the total volume of the liquid, of water. In some embodiments, the solvent is an aqueous ethereal solution, such as diethyl ether, dimethyl ether, methyl ethyl ether, diphenyl ether, or dipropyl ether, or water, among others. Preferably, the solvent is water.

Desirably, the reaction is performed at an elevated temperature, i.e., above room temperature. In some embodiments, the reaction is performed at a temperature of at least about 30° C., about 35° C., about 40° C., about 45° C., about 50° C., about 55° C., or about 60° C. Preferably, the reaction is performed at about 40 to about 55° C., about 40 to about 50° C., about 40 to about 45° C., about 45 to about 55° C., about 45 to about 50° C., about 50 to about 55° C. More preferably, the reaction is performed at about 45 to about 55° C. The reaction is desirably performed for a sufficient period of time to convert the compound of formula II to the compound of formula I. In some embodiments, the reaction is performed for at least about 1 day, or at least about 2, about 3, about 4, about 5, about 6, or about 7 days, preferably at least about 2 days. The compound of formula I may be isolated using techniques known to those of skill in the art. In some embodiments, the compound of formula I is isolated using neutralization. One skilled in the art would be able to select a suitable base for the neutralization from among, without limitation, hydroxide bases such as ammonium hydroxide, sodium hydroxide, potassium hydroxide, or lithium hydroxide, among others, or combinations thereof.

The compounds of formula II may be prepared from compounds of formula III.

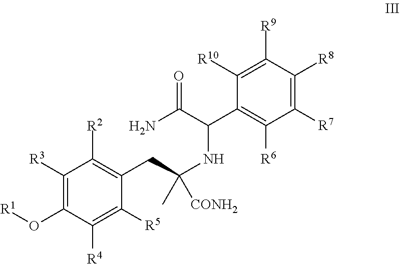

In the compounds of formula III, R¹ to R⁵ are defined above and R⁶ to R¹⁰ are, independently, H, halo, C₁₋₆alkyl, C₂₋₆alkenyl, NR¹¹R¹², OH, C₁₋₆alkoxy, or aryl; and R¹¹ and R¹² are, independently, H or C₁₋₆alkyl. In some embodiments, R⁶ is H, halo, C₁₋₆alkyl, C₂₋₆alkenyl, NR¹¹R¹², OH, C₁₋₆alkoxy, or aryl. In other embodiments, R⁶ is H. In further embodiments, R⁶ is halo such as F, Cl, or Br. In further embodiments, R⁶ is C₁₋₆alkyl such as methyl, ethyl, propyl, butyl, pentyl, or hexyl. In still other embodiments, R⁶ is C₂₋₆alkenyl such as ethenyl, propenyl, butenyl, pentenyl, or hexenyl. In further embodiments, R⁶ is NR¹¹R¹² such as NH₂ or N(C₁₋₆alkyl)(C₁₋₆alkyl). In yet other embodiments, R⁶ is OH. In still further embodiments, R⁶ is C₁₋₆alkoxy such as methoxy, ethoxy, propoxy, butoxy, pentoxy, or hexoxy. In further embodiments, R⁶ is aryl such as phenyl.

In some embodiments, R⁷ is H, halo, C₁₋₆alkyl, C₂₋₆alkenyl, NR¹¹R¹², OH, C₁₋₆alkoxy, or aryl. In other embodiments, R⁷ is H. In further embodiments, R⁷ is halo such as F, Cl, or Br. In further embodiments, R⁷ is C₁₋₆alkyl such as methyl, ethyl, propyl, butyl, pentyl, or hexyl. In still other embodiments, R⁷ is C₂₋₆alkenyl such as ethenyl, propenyl, butenyl, pentenyl, or hexenyl. In further embodiments, R⁷ is NR¹¹R¹² such as NH₂ or N(C₁₋₆alkyl)(C₁₋₆alkyl). In yet other embodiments, R⁷ is OH. In still further embodiments, R⁷ is C₁₋₆alkoxy such as methoxy, ethoxy, propoxy, butoxy, pentoxy, or hexoxy. In further embodiments, R⁷ is aryl such as phenyl.

In some embodiments, R⁸ is H, halo, C₁₋₆alkyl, C₂₋₆alkenyl, NR¹¹R¹², OH, C₁₋₆alkoxy, or aryl. In other embodiments, R⁸ is H. In further embodiments, R⁸ is halo such as F, Cl, or Br. In further embodiments, R⁸ is C₁₋₆alkyl such as methyl, ethyl, propyl, butyl, pentyl, or hexyl. In still other embodiments, R⁸ is C₂₋₆alkenyl such as ethenyl, propenyl, butenyl, pentenyl, or hexenyl. In further embodiments, R⁷ is NR¹¹R¹² such as NH₂ or N(C₁₋₆alkyl)(C₁₋₆alkyl). In yet other embodiments, R⁸ is OH. In still further embodiments, R⁸ is C₁₋₆alkoxy such as methoxy, ethoxy, propoxy, butoxy, pentoxy, or hexoxy. In further embodiments, R⁸ is aryl such as phenyl.

In some embodiments, R⁹ is H, halo, C₁₋₆alkyl, C₂₋₆alkenyl, NR¹¹R¹², OH, C₁₋₆alkoxy, or aryl. In other embodiments, R⁹ is H. In further embodiments, R⁹ is halo such as F, Cl, or Br. In further embodiments, R⁹ is C₁₋₆alkyl such as methyl, ethyl, propyl, butyl, pentyl, or hexyl. In still other embodiments, R⁹ is C₂₋₆alkenyl such as ethenyl, propenyl, butenyl, pentenyl, or hexenyl. In further embodiments, R⁹ is NR¹¹R¹² such as NH₂ or N(C₁₋₆alkyl)(C₁₋₆alkyl). In yet other embodiments, R⁹ is OH. In still further embodiments, R⁹ is C₁₋₆alkoxy such as methoxy, ethoxy, propoxy, butoxy, pentoxy, or hexoxy. In further embodiments, R⁹ is aryl such as phenyl.

In some embodiments, R¹⁰ is H, halo, C₁₋₆alkyl, C₂₋₆alkenyl, NR¹¹R¹², OH, C₁₋₆alkoxy, or aryl. In other embodiments, R¹⁰ is H. In further embodiments, R¹⁰ is halo such as F, Cl, or Br. In further embodiments, R¹⁰ is C₁₋₆alkyl such as methyl, ethyl, propyl, butyl, pentyl, or hexyl. In still other embodiments, R¹⁰ is C₂₋₆alkenyl such as ethenyl, propenyl, butenyl, pentenyl, or hexenyl. In further embodiments, R¹⁰ is NR¹¹R¹² such as NH₂ or N(C₁₋₆alkyl)(C₁₋₆alkyl). In yet other embodiments, R¹⁰ is OH. In still further embodiments, R¹⁰ is C₁₋₆alkoxy such as methoxy, ethoxy, propoxy, butoxy, pentoxy, or hexoxy. In further embodiments, R¹⁰ is aryl such as phenyl.

Preferably, at least one of R⁶ to R¹⁰ is H, and, more preferably, all of R⁶ to R¹⁰ are H. In some embodiments, the compound of formula III is compound 3:

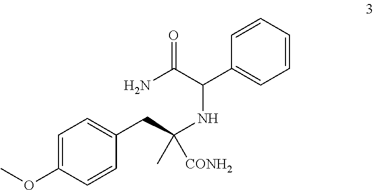 [SMILES: COc1ccc(C[C@@](C)(NC(C(N)=O)c2ccccc2)C(N)=O)cc1]

The compound of formula III may be converted to the compound of formula II by hydrogenating a compound of formula III in a solvent and at a temperature sufficient to prepare the compound of formula II or a solvate thereof. In some embodiments, the hydrogenation is performed using a palladium catalyst and a hydrogen source. The palladium catalyst may be selected my one skilled in the art from among Pd/C, palladium acetate, Pd(OAc)₂, tetrakis(triphenylphosphine)palladium(0), Pd(PPh₃)₄, bis(triphenylphosphine)palladium(II) dichloride, PdCl₂(PPh₃)₂, or [1,1′-bis(diphenylphosphino)ferrocene]palladium(II) dichloride, among others. The palladium catalyst may be added in one aliquot or two or more aliquots, such as 2, 3, 4, or 5 aliquots, preferably 2 aliquots. The term “hydrogen source” as used herein refers to a reagent that supplies hydrogen atoms. In some embodiments, the hydrogen source is hydrogen gas, ethene, propene, butene, or an acid such as formic acid, ethanolic acid, propanoic acid, or butanoic acid, among others, or combinations thereof. Preferably the hydrogen source is formic acid. The hydrogen source may be added in one aliquot or two or more aliquots, such as 2, 3, 4, or 5 aliquots, preferably 2 aliquots. In certain embodiments, the reaction comprises a single aliquot of the palladium catalyst, single aliquot of the hydrogen source, preferably formic acid, or a combination thereof. Desirably, an excess of the hydrogen source is utilized for the hydrogenation. In certain embodiments, at least about 2, at least about 3, at least about 4, at least about 5, at least about 6, at least about 7, at least about 8, at least about 9, at least about 10, at least about 20, at least about 25, or at least about 50 equivalents of the hydrogen source are added. Desirably, the reaction is performed at an elevated temperature, i.e., above room temperature. The hydrogenation is performed at a temperature of at least about 30° C., about 35° C., about 40° C., about 45° C., about 50° C., about 55° C., or about 60° C. Preferably, the hydrogenation is performed at about 40 to about 55, about 40 to about 50° C., about 40 to about 45° C., about 45 to about 55° C., about 45 to about 50° C., about 50 to about 55° C. More preferably, the hydrogenation is performed at about 45 to about 55° C. The hydrogenation is performed in an alcoholic solvent, such as methanol, ethanol, propanol, butanol, among others, or combinations thereof. Preferably, the alcoholic solvent is methanol.

The compound of formula III may be prepared by reacting a compound of formula IV with a hydrolyzing agent. The compound of formula IV has the following structure, wherein R¹ to R¹⁰ are defined herein.

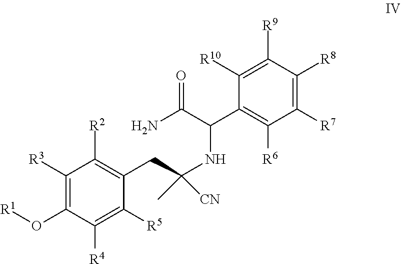

In certain embodiments, the compound of formula IV is compound 4:

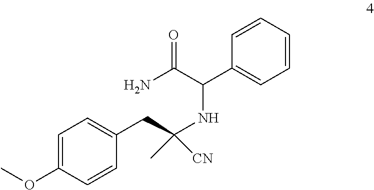 [SMILES: COc1ccc(C[C@](C)(C#N)NC(C(N)=O)c2ccccc2)cc1]

The hydrolyzing agent is an acid, base, hydroperoxide, or an enzyme. In some embodiments, the hydrolyzing agent is an acid, such as sulfuric acid, a sulfonic acid such as CF₃SO₃H, a hydrogen halide such as hydrochloric acid, hydrobromic acid, or hydroiodic acid, acetic acid, or polyphosphoric acid, preferably sulfuric acid. In other embodiments, the hydrolyzing agent is a base such as an amine such as ammonia, diethylamine, or methylamine or sodium bicarbonate, among others. In further embodiments, the hydrolyzing agent is a peroxide such as hydroperoxide, acetyl acetone peroxide, tert-butyl hydroperoxide, or diacetyl peroxide, among others, or combinations thereof. Preferably, the hydrolyzing agent is hydroperoxide. In still other embodiments, the hydrolyzing agent is an enzyme such as a protease, amylase, or lipase. The hydrolyzing agent is added at a rate that controls the reaction, as determined by one of skill in the art. Desirably, the hydrolyzing agent is added at a rate that controls the exothermic reaction. In some embodiments, the hydrolyzing agent is added dropwise or over a period of time. In other embodiments, the hydrolyzing agent is added over a period of at least about 1 minute, at least about 5 minutes, at least about 10 minutes, at least about 30 minutes, or at least about 60 minutes. The hydrolyzing may be performed in a solvent such as dichloromethane and/or at temperatures before room temperature. Preferably, the hydrolyzing is performed at about −25 to about 25° C., more preferably about −10 to about 10° C., or most preferably about 0 to about 10° C. The reaction may be performed in a solvent that is miscible with the acid including, without limitation, dichloromethane, alcoholic solvents such as methanol, ethyl acetate, propane-2-one, cyclopentane or 2-metnyl tetrahydrofuran, among others, or combinations such as ethyl acetate/ethanol or propan-2-one/cyclopentane. Preferably, the solvent is dichloromethane.

The compound of formula IV is prepared by (a) reacting a compound of formula V with a compound of formula IV in the presence of an acid; and (b) reacting the product of step (a) with a cyanide source, wherein R¹ to R¹⁰ are defined herein.

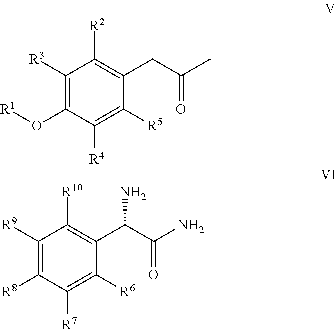

In some embodiments, the compound of formula V is compound 5. In other embodiments, the compound of formula VI is compound 6. In further embodiments, the compound of formula V is compound 5 and the compound of formula VI is compound 6.

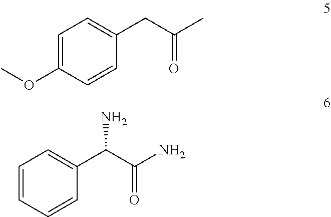

In the preparation of the compound of formula IV, step (a) is performed using an acid. In some embodiments, the acid is an aqueous acid in a solvent and at a temperature sufficient for at least about 48 hours. In some embodiments, the aqueous acid is an aqueous hydrogen halide, such as hydrogen chloride, hydrogen bromide, or hydrogen iodide. Preferably, the aqueous acid is hydrogen chloride. Step (a) may be performed at a temperature below about ambient or room temperatures, i.e., below about 25° C. Preferably, step (a) is performed at about −25 to about 25° C., more preferably about −10 to about 10° C., or most preferably about 0 to about 10° C. The reaction may be performed in a solvent that is miscible with the acid. In some embodiments, the step (a) solvent is an aqueous solvent, preferably an aqueous alcoholic solvent, such as methanol, ethanol, propanol, butanol, among others. Preferably, the alcoholic solvent is methanol.

Step (b) to the formation of the compound of formula IV comprises adding a cyanide source to the product of step (a). In some embodiments, the cyanide source is sodium cyanide or potassium cyanide, preferably sodium cyanide. Step (b) is desirably performed at elevated temperatures, such as above room or ambient temperature. In some embodiments, step (b) is performed at a temperature of at least about 25° C., i.e., at least about 30° C., about 35° C., about 40° C., about 45° C., about 50° C., or about 55° C., preferably at least about 40° C.

Additional purification steps may be performed to purify one or more compounds described herein. In some embodiments, the compound of formula I is purified, preferably compound 1 is purified. In other embodiments, the compound of formula II is purified. In further embodiments, the compound of formula III is purified. In still other embodiments, the compound of formula IV is purified.

The purification of compound I may be performed by converting the compound of formula I to a salt of the compound of formula I, then converting the compound I salt to purified compound I. Conversion of compound I to the compound I salt is performed using an aqueous acid. In some embodiments, the aqueous acid is a hydrogen halide, such as hydrochloric acid, hydrobromic acid, or hydroiodic acid, preferably hydrochloric acid. The salt may be formed using elevated temperatures, i.e., a temperature above room temperature. The reaction is performed in an aqueous solvent, such as water. In some embodiments, the reaction is performed at a temperature of at least about 25° C., about 30° C., about 40° C., about 50° C., about 60° C., or about 70° C., preferably at least about 50° C. The reaction is maintained at elevated temperatures for a period of time sufficient to form the compound I salt. In some embodiments, the reaction is maintained for at least about 10 minutes, about 20 minutes, about 30 minutes, about 40 minutes, about 50 minutes, or about 60 minutes, preferably at least about 30 minutes. Once the salt has formed, the reaction solution is optionally cooled. In some embodiments, the reaction solution is cooled to about 10 to about 30° C., preferably about 10 to about 25° C., or more preferably about 15 to about 25° C., or even more preferably about 20° C. Once formed, the compound I salt may be converted back to the compound of formula I. In certain embodiments, the conversion the compound I salt to the neutral compound of formula I is performed by crystallizing the purified compound of formula I, preferably by crystallizing purified D-metyrosine. In some embodiments, the purified compound I is prepared by adjusting the pH to about 4 to about 7. In some embodiments, the pH is adjusted to about 4, about 4.5, about 5, about 5.5, about 6, about 6.5, or about 7. Preferably, the pH is adjusted to about 5 to about 6. The pH may be adjusted using conditions suitable to convert an acid salt to a neutral compound. In some embodiments, the pH is adjusted using a base such as a hydroxide base, such as sodium hydroxide, potassium hydroxide, or ammonium hydroxide, preferably ammonium hydroxide. The pH may be adjusted at elevated temperatures such as at least about 40° C. In some embodiments, the pH is adjusted at a temperature of about 40° C., about 45° C., about 50° C., about 55° C., about 60° C., about 65° C., or about 70° C., preferably about 40 to about 60° C., or preferably about 45 to about 55° C.

The purification of compound III may be performed by crystallization. The crystallization may be performed using an aqueous solvent. In some embodiments, the solvent is water. In other embodiments, the solvent contains water and another solvent that is miscible with water, such as methylisobutyl ketone, acetic acid, acetone, acetonitrile, N-methyl-2-pyrrolidone, among others, or combinations thereof. Preferably, the solvent is water/methylisobutyl ketone. The crystallization may be performed at elevated temperatures such as at least about 40° C. In some embodiments, the pH is adjusted at a temperature of about 40° C., about 45° C., about 50° C., about 55° C., about 60° C., about 65° C., about 70° C., about 75° C., about 80° C., about 85° C., or about 90° C., preferably about 60 to about 90° C., or more preferably about 70 to about 80° C. The elevated temperature is typically maintained for a period of time as determined by one of skill in the art and then the solution is cooled. In some embodiments, the solution is cooled to a temperature that is below about room temperature, such as about −20 to about 20° C. In some embodiments, the solution is cooled to about −20° C., about −15° C., about −10° C., about 5° C., about 0° C., about 5° C., about 10° C., about 15° C., or about 20° C., preferably about −5 to about 10° C., or more preferably about 0 to about 5° C.

Compositions Containing D-Metyrosine

Pharmaceutical compositions useful herein, in some embodiments, contain one or more compounds of formula I, such as D-metyrosine, in a pharmaceutically acceptable carrier or diluent with other optional suitable pharmaceutically inert or inactive ingredients. In some embodiments, one or more compounds of formula I, such as D-metyrosine, is present in a single composition. In further embodiments, one or more compounds of formula I, such as D-metyrosine, is combined with one or more excipients and/or other therapeutic agents as described below. In certain embodiments, the D-metyrosine is prepared as described herein.

The compositions described herein may contain varying amounts of the containing one or more compounds of formula I. Thus, in some embodiments, the compositions contain varying amounts of D-metyrosine. In certain embodiments, the composition contains at least about 10 wt %, based on the weight of the composition, of D-metyrosine. In other embodiments, the composition contains at least about 20 wt %, at least about 30 wt %, at least about 40 wt %, at least about 50 wt %, at least about 60 wt %, at least about 70 wt %, at least about 80 wt %, at least about 90 wt %, or about 100 wt %, based on the weight of the composition, of D-metyrosine. In other embodiments, the composition contains about 10 to about 90 wt % of D-metyrosine, about 10 to about 80 wt %, about 10 to about 70 wt %, about 10 to about 60 wt %, about 10 to about 50 wt %, about 10 to about 40 wt %, about 10 to about 30 wt %, about 10 to about 20 wt %, about 20 to about 90 wt %, about 20 to about 80 wt %, about 20 to about 70 wt %, about 20 to about 60 wt %, about 20 to about 50 wt %, about 20 to about 40 wt %, about 20 to about 30 wt %, about 30 to about 90 wt %, about 30 to about 80 wt %, about 30 to about 70 wt %, about 30 to about 60 wt %, about 30 to about 50 wt %, about 30 to about 40 wt %, about 40 to about 90 wt %, about 40 to about 80 wt %, about 40 to about 70 wt %, about 40 to about 60 wt %, or about 40 to about 50 wt %, based on the weight of the composition, of D-metyrosine. In further embodiments, the composition contains about 50 to about 99 wt %, based on the weight of the composition, of D-metyrosine. In yet other embodiments, the composition contains about 50 to about 90 wt %, about 50 to about 80 wt %, about 50 to about 70 wt %, about 50 to about 60 wt %, about 60 to about 99 wt %, about 60 to about 90 wt %, about 60 to about 80 wt %, about 60 to about 70 wt %, about 70 to about 99 wt %, about 70 to about 90 wt %, about 70 to about 80 wt %, about 80 to about 99 wt %, about 90 to about 99 wt %, based on the weight of the composition of D-metyrosine. In still further embodiment, the composition contains at least about 50, 55, 60, 65, 70, 75, 80, 85, 90, 95, or 99 wt %, based on the weight of the composition, of D-metyrosine.

The compositions may also be mixtures containing D-metyrosine and L-metyrosine. In some embodiments, the mixture contains L-metyrosine and at least about 10 wt %, based on the weight of the composition, of D-metyrosine. In further embodiments, the mixture contains L-metyrosine and at least about 15 wt %, at least about 20 wt %, at least about 25 wt %, at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, at least about 80 wt %, at least about 85 wt %, at least about 90 wt %, at least about 95 wt %, or at least about 99 wt %, based on the weight of the composition, of D-metyrosine. In some embodiments, the mixture contains D-metyrosine and at least about 10 wt %, based on the weight of the composition, of L-metyrosine. In other embodiments, the mixture contains D-metyrosine and at least about 15 wt %, at least about 20 wt %, at least about 25 wt %, at least about 30 wt %, at least about 35 wt %, at least about 40 wt %, at least about 45 wt %, at least about 50 wt %, at least about 55 wt %, at least about 60 wt %, at least about 70 wt %, at least about 80 wt %, at least about 90 wt %, or about 100 wt %, based on the weight of the composition, of L-metyrosine.

In still other embodiments, the mixture contains about 10 wt % of D-metyrosine and about 90 wt % of L-metyrosine, about 15 wt % of D-metyrosine and about 85 wt % of L-metyrosine, about 20 wt % of D-metyrosine and about 80 wt % of L-metyrosine, about 25 wt % of D-metyrosine and about 75 wt % of L-metyrosine, about 30 wt % of D-metyrosine and about 70 wt % of L-metyrosine, about 35 wt % of D-metyrosine and about 65 wt % of L-metyrosine, about 40 wt % of D-metyrosine and about 60 wt % of L-metyrosine, about 45 wt % of D-metyrosine and about 55 wt % of L-metyrosine, about 55 wt % of D-metyrosine and about 45 wt % of L-metyrosine, about 60 wt % of D-metyrosine and about 40 wt % of L-metyrosine, about 65 wt % of D-metyrosine and about 35 wt % of L-metyrosine, about 70 wt % of D-metyrosine and about 30 wt % of L-metyrosine, about 75 wt % of D-metyrosine and about 25 wt % of L-metyrosine, about 80 wt % of D-metyrosine and about 20 wt % of L-metyrosine, about 85 wt % of D-metyrosine and about 15 wt % of L-metyrosine, or about 90 wt % of D-metyrosine and about 10 wt % of L-metyrosine.

(i) Salts

The compounds of formula I, such as D-metyrosine prepared as described herein, may encompass tautomeric forms of the structures provided herein characterized by the bioactivity of the drawn structures. Further, any of the compounds described herein, including the compounds of formula I, formula II, formula III, formula IV, formula V, or formula VI, or compound 1, compound 2, compound 3, compound 4, compound 5, or compound 6, may be isolated or used in the form of salts derived from pharmaceutically or physiologically acceptable acids, bases, alkali metals and alkaline earth metals.

In some embodiments, pharmaceutically acceptable salts can be formed from organic and inorganic acids including, e.g., acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids.

In other embodiments, pharmaceutically acceptable salts may also be formed from inorganic bases, desirably alkali metal salts including, e.g., sodium, lithium, or potassium, such as alkali metal hydroxides. Examples of inorganic bases include, without limitation, sodium hydroxide, potassium hydroxide, calcium hydroxide, and magnesium hydroxide. Pharmaceutically acceptable salts may also be formed from organic bases, such as ammonium salts, mono-, di-, and trimethylammonium, mono-, di- and triethylammonium, mono-, di- and tripropylammonium, ethyldimethylammonium, benzyldimethylammonium, cyclohexylammonium, benzyl-ammonium, dibenzylammonium, piperidinium, morpholinium, pyrrolidinium, piperazinium, 1-methylpiperidinium, 4-ethylmorpholinium, 1-isopropylpyrrolidinium, 1,4-dimethylpiperazinium, 1 n-butyl piperidinium, 2-methylpiperidinium, 1-ethyl-2-methylpiperidinium, mono-, di- and triethanolammonium, ethyl diethanolammonium, n-butylmonoethanolammonium, tris(hydroxymethyl)methylammonium, phenylmono-ethanolammonium, diethanolamine, ethylenediamine, and the like. In one example, the base is selected from among sodium hydroxide, lithium hydroxide, potassium hydroxide, and mixtures thereof.

(ii) Prodrugs

The salts, as well as other compounds prepared as described herein, can be in the form of esters, carbamates and other conventional “pro-drug” forms, which, when administered in such form, convert to the active moiety in vivo. In some embodiments, the prodrugs are esters. In other embodiments, the prodrugs are carbamates. See, e.g., B. Testa and J. Caldwell, “Prodrugs Revisited: The “Ad Hoc” Approach as a Complement to Ligand Design”, Medicinal Research Reviews, 16(3):233-241, ed., John Wiley & Sons (1996), which is incorporated by reference.

(iii) Carriers and Diluents

The pharmaceutical compositions include one or more compounds of formula I, such as D-metyrosine prepared as described herein, formulated neat or with one or more pharmaceutical carriers for administration, the proportion of which is determined by the solubility and chemical nature of the compound, chosen route of administration and standard pharmacological practice. The pharmaceutical carrier may be solid or liquid.

Although the compound of formula I, such as D-metyrosine prepared as described herein, may be administered alone, it may also be administered in the presence of one or more pharmaceutical carriers that are physiologically compatible. The carriers may be in dry or liquid form and must be pharmaceutically acceptable. Liquid pharmaceutical compositions are typically sterile solutions or suspensions.

When liquid carriers are utilized, they are desirably sterile liquids. Liquid carriers are typically utilized in preparing solutions, suspensions, emulsions, syrups and elixirs. In one embodiment, the compound of formula I, such as D-metyrosine prepared as described herein, is dissolved a liquid carrier. In another embodiment, the compound is suspended in a liquid carrier. One of skill in the art of formulations would be able to select a suitable liquid carrier, depending on the route of administration. In one embodiment, the liquid carrier includes, without limitation, water, organic solvents, oils, fats, or mixtures thereof. In another embodiment, the liquid carrier is water containing cellulose derivatives such as sodium carboxymethyl cellulose. In a further embodiment, the liquid carrier is water and/or dimethylsulfoxide. Examples of organic solvents include, without limitation, alcohols such as monohydric alcohols and polyhydric alcohols, e.g., glycols and their derivatives, among others. Examples of oils include, without limitation, fractionated coconut oil, arachis oil, corn oil, peanut oil, and sesame oil and oily esters such as ethyl oleate and isopropyl myristate.

Alternatively, the compound of formula I, such as D-metyrosine prepared as described herein, may be formulated in a solid carrier. In one embodiment, the composition may be compacted into a unit dose form, i.e., tablet or caplet. In another embodiment, the composition may be added to unit dose form, i.e., a capsule. In a further embodiment, the composition may be formulated for administration as a powder. The solid carrier may perform a variety of functions, i.e., may perform the functions of two or more of the excipients described below. For example, the solid carrier may also act as a flavoring agent, lubricant, solubilizer, suspending agent, filler, glidant, compression aid, binder, disintegrant, or encapsulating material. Suitable solid carriers include, without limitation, calcium phosphate, dicalcium phosphate, magnesium stearate, talc, starch, sugars (including, e.g., lactose and sucrose), cellulose (including, e.g., microcrystalline cellulose, methyl cellulose, sodium carboxymethyl cellulose), polyvinylpyrrolidine, low melting waxes, ion exchange resins, and kaolin. The solid carrier can contain other suitable excipients, including those described below.

Examples of excipients which may be combined with the compound of formula I, such as D-metyrosine prepared as described herein, include, without limitation, adjuvants, antioxidants, binders, buffers, coatings, coloring agents, compression aids, diluents, disintegrants, emulsifiers, emollients, encapsulating materials, fillers, flavoring agents, glidants, granulating agents, lubricants, metal chelators, osmo-regulators, pH adjustors, preservatives, solubilizers, sorbents, stabilizers, sweeteners, surfactants, suspending agents, syrups, thickening agents, or viscosity regulators. See, the excipients described in the “Handbook of Pharmaceutical Excipients”, 5th Edition, Eds.: Rowe, Sheskey, and Owen, APhA Publications (Washington, D.C.), Dec. 14, 2005, which is incorporated herein by reference.

In the following example, efforts have been made to ensure accuracy with respect to numbers used (e.g., amounts, temperature, etc.) but some experimental error and deviation should be accounted for. Unless indicated otherwise, temperature is in degrees C., pressure is at or near atmospheric.

### EXAMPLES

### Example 1

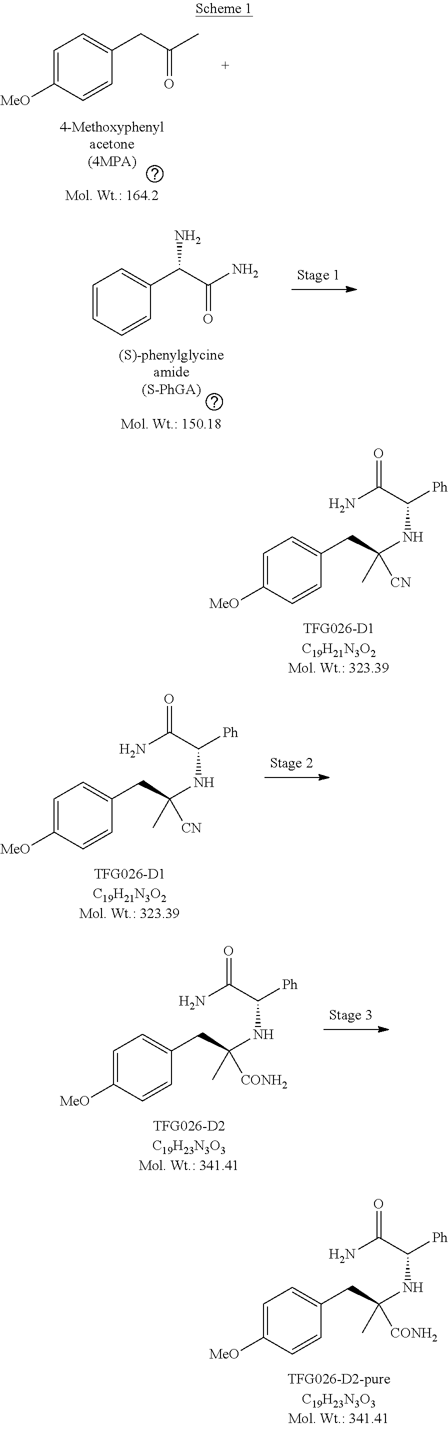 [SMILES: COc1ccc(CC(C)=O)cc1 ; NC(=O)[C@@H](N)c1ccccc1 ; COc1ccc(C[C@](C)(C#N)N[C@H](C(N)=O)c2ccccc2)cc1 ; COc1ccc(C[C@](C)(C#N)N[C@H](C(N)=O)c2ccccc2)cc1 ; COc1ccc(C[C@@](C)(N[C@H](C(N)=O)c2ccccc2)C(N)=O)cc1 ; COc1ccc(C[C@@](C)(N[C@H](C(N)=O)c2ccccc2)C(N)=O)cc1]

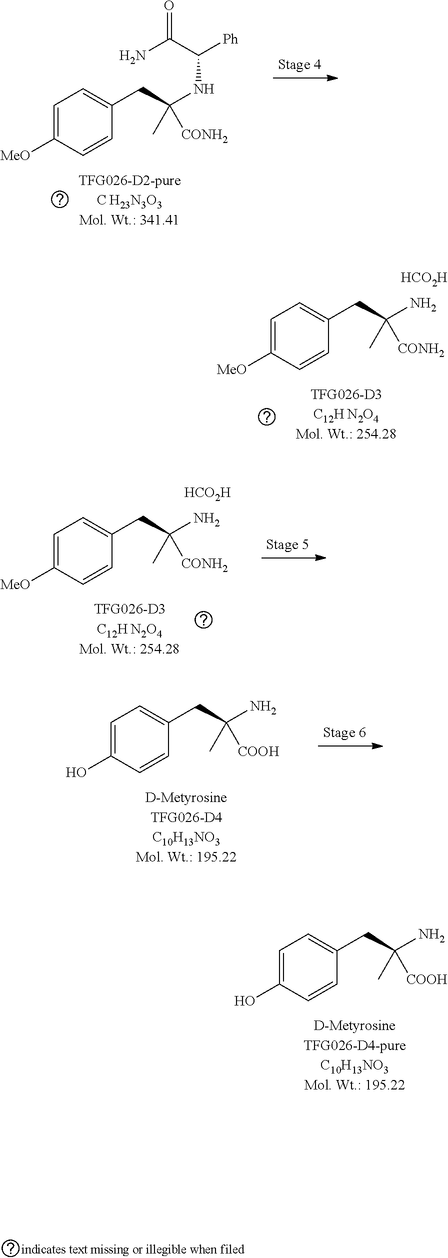

### Step 1: Preparation of TFG026-D1 (25 g Scale)

To a clean reactor is charged S-PhGA (1.0 eq.), 4MPA (1.0 eq.), methanol (3.8 vol) and water (6.9 vol). The reactor contents were cooled to 5±5° C. and conc. HCl (1.0 eq.) was then charged while maintaining the internal temperature of ≤20° C. After rinsing the residual HCl forward with a small amount of water (˜0.3 vol), NaCN (1.0 eq.) was charged portion wise over 15 minutes while maintaining an internal temperature of ≤20° C. The residual NaCN was rinsed forward with water (˜0.3 vol) and the reactor contents were warmed to 45±5° C. After 34 hours, an IPC sample was pulled for HPLC analysis. The reaction was cooled to 20±5° C. and was aged for ≥1.5 hours. The contents were filtered and the cake was washed with 7:3 (v/v) water: methanol (2×1.9 vol) followed by 2-propanol (2×1.7 vol). The solids were then dried under vacuum at ≤45° C. to provide TFG026-D1 (77%). Note: All calculations are based off of 4MPA

### Step 2: Preparation of TFG026-D2 (32 g Scale)

To a clean reactor was charged TFG026-D1 (1.0 eq.) and DCM (10 vol), which was subsequently cooled to 0±5° C. Conc. Sulfuric acid (1.2 mass eq.) was charged at a constant rate over 1 hour while maintaining an internal temperature of 5±5° C. An IPC sample for HPLC analysis was immediately pulled to determine reaction completion. Water (20 vol) was then charged to the reaction mixture over 2 hours, while maintaining the internal temperature ≤25° C. The reaction mixture was then aged for ≥1 hour at 20±5° C. The layers were separated and the organic layer was washed with water (1 vol). The combined organic layers were washed with DCM (1 vol). The combined aqueous layers were then distilled at ≥30° C. to remove any residual DCM. The aqueous layers were then cooled to 5±5° C. and were treated with 28-30% ammonium hydroxide (3 vol) over 1.5 hours while maintaining the internal temperature ≤25° C. The cake was filtered and washed with water (2×5 vol) and the solids were dried under vacuum (≤45° C.) to give TFG026-D2 (89%).

### Step 3: Preparation of TFG026-D2 Pure (32 g Scale)

To a clean reactor was charged TFG026-D2 (1.0 eq.), water (1 vol) and MIBK (10 vol). The slurry was then agitated at 75±5° C. to obtain a clear solution. The reaction mixture was then cooled to 0-5° C. and aged for ≥1 hour. The resulting slurry was filtered and the cake was washed with MIBK (2×1 vol) and the solids were dried at 35° C. under vacuum to give purified TFG026-D2 (95%).

### Step 4: Preparation of TFG026-D3 (30 g Scale)

To a clean reactor was charged TFG026-D2-pure (1 eq.), 10% Pd/Carbon (wet) (5 wt %), and MeOH (5.7 vol). The reactor contents were warmed to 55±5° C. and a solution of formic acid (7.5 eq.) in water (1.5 vol) was added over ≥30 minutes to maintain an internal temperature of 55±5° C. After aging for ≥1 hour, an IPC sample was pulled for HPLC analysis to determine reaction completion. The reaction mixture was cooled to 20±5° C. and subsequently filtered. The catalyst was washed with MeOH (3×1 vol). The combined filtrates were concentrated to −2 vol and water (0.6 vol) was charged. This concentration procedure was repeated twice. DCM (10 vol) was then charged and the layers were separated. The aqueous layer was then washed with more DCM (3×5 vol). The aqueous phase was concentrated to remove residual DCM and was then lyophilized to TFG026-D3 (62%).

### Step 5: Preparation of TFG026-D4 (18 g Scale)

To a clean reactor was charged TFG026-D4 (1.0 eq.), water (3 vol) and 48% HBr (11 eq.). The reaction mixture was then heated to reflux and agitated for ˜2 days before the reaction was deemed complete via HPLC. The reactor contents were then cooled to 50-55° C. and was neutralized to pH=6 with ammonium hydroxide. The slurry was cooled further to 20±5° C. and aged for ≥3 hours. The reaction mixture was filtered and the cake was washed with water (3×3 vol) followed by 2-propanol (2.5 vol, then 1.5 vol). The solids were vacuum-dried to give TFG026-D4 (53%).

### Step 6: Preparation of Purified D-a-Methyltyrosine (10 g Scale)

To a glass reactor is charged TFG026-D4, 6N HCl (aq.) (4.0V) and water (3V). The slurry was then heated to 50±5° C. Upon dissolution, the reaction mixture was allowed to age for ≥30 minutes. The heat was turned off and the reaction was allowed to cool to 20±5° C. The pH was then adjusted to 5-6 at 50±5° C. via 28-30% ammonium hydroxide. The slurry is then cooled to 10±5° C. and aged for 1 hour. The solids are then isolated via filtration and the cake is washed with water (4V) to give purified TFG026-D4 (89%).

The contents of all references, patent applications, patents, and published patent applications, as well as the Figures, cited throughout this application are hereby incorporated by reference.

Those skilled in the art will recognize, or be able to ascertain using no more than routine experimentation, many equivalents to the specific embodiments of the disclosure described herein. Such equivalents are intended to be encompassed by the following claims.

1. A process for preparing a compound of formula I, comprising:
reacting a compound of formula II for at least about 48 hours with an aqueous acid in a solvent and at a temperature sufficient to produce a compound of formula I: 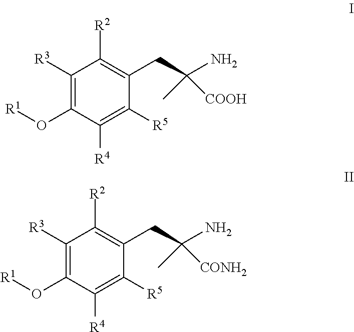
wherein:
R¹ is C₁₋₆alkyl, C₃₋₈cycloalkyl, or aryl; and
R² to R⁵ are, independently, H, halo, C₁₋₆alkyl, C₁₋₆alkoxy, C₃₋₈cycloalkyl, or aryl.

2. The process of claim 1, wherein the acid is an aqueous hydrogen halide, such as hydrogen bromide.

3. The process of claim 1, wherein the solvent is an aqueous solvent, such as water.

4. The process of claim 1, wherein the temperature of the solvent is at least about 45° C., preferably at least about 50° C.

5. The process of claim 1, wherein R¹ is C₁₋₆alkyl, preferably methyl.

6. The process of claim 1, wherein one or more of R² to R⁵ is H, preferably R² to R⁵ all are H.

7. The process of claim 1, wherein the compound of formula I is D-metyrosine (compound 1):

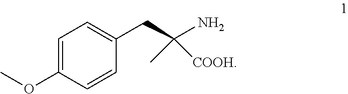

8. The process of claim 1, wherein the compound of formula II is compound 2:

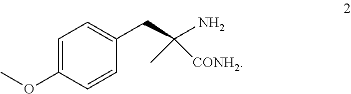

9. The process of claim 1, further comprising hydrogenating a compound of formula III in a solvent and at a temperature sufficient to prepare the compound of formula II or a solvate thereof: 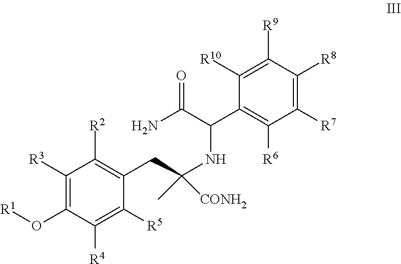
wherein:
R⁶ to R¹⁰ are, independently, H, halo, C₁₋₆alkyl, C₂₋₆alkenyl, NR¹¹R¹², OH, C₁₋₆alkoxy, or aryl;
R¹¹ and R¹² are, independently, H or C₁₋₆alkyl.

10. The process of claim 9, wherein the hydrogenation is performed using a palladium catalyst, such as Pd/C, and a hydrogen source, such as hydrogen gas or formic acid, preferably formic acid.

11. (canceled)

12. The process of claim 9, comprising at least about 1.1 equivalents of the formic acid.

13. The process of claim 9, wherein at least one of R⁶ to R¹⁰ is H, preferably all of R⁶ to R¹⁰ are H.

14. The process of claim 9, wherein the compound of formula III is compound 3:

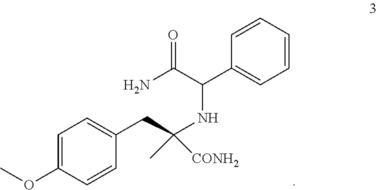 [SMILES: COc1ccc(C[C@@](C)(NC(C(N)=O)c2ccccc2)C(N)=O)cc1]

15. The process of claim 9, further comprising reacting a compound of formula IV with a hydrolyzing agent to prepare the compound of formula III:

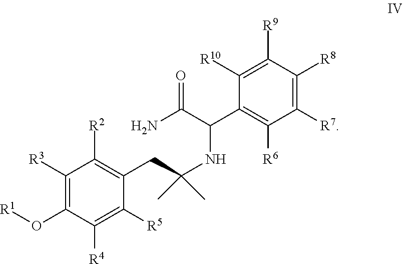

16. The process of claim 15, wherein the hydrolyzing agent is an acid, base, hydroperoxide, or an enzyme, preferably an acid.

17. The process of claim 16, wherein the acid is an aqueous acid such as sulfuric acid.

18. The process of claim 17, wherein the aqueous acid is added to the compound of formula IV, preferably at a rate that controls the exothermic reaction.

19. The process of claim 15, wherein the compound of formula IV is compound 4:

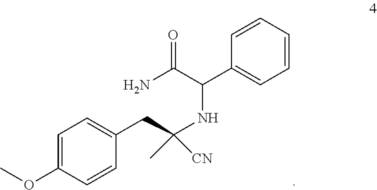 [SMILES: COc1ccc(C[C@](C)(C#N)NC(C(N)=O)c2ccccc2)cc1]

20. The process of claim 15, further comprising preparing the compound of formula IV by:
(a) reacting a compound of formula V with a compound of formula IV in the presence of an acid; 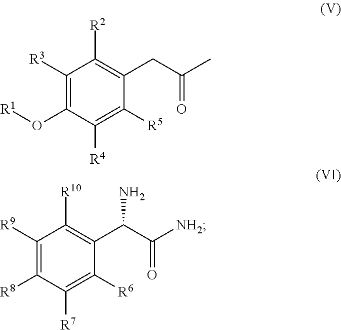
and
(b) reacting the product of step (b) with a cyanide source.

21. The process of claim 20, wherein the cyanide source is sodium cyanide or potassium cyanide, preferably sodium cyanide.

22. The process of claim 20, wherein step (a) is performed at a temperature of less than about ambient temperature.

23. The process of claim 20, wherein step (b) is performed at elevated temperature, such as at least about 40° C.

24. The process of claim 20, wherein the compound of formula IV is prepared at a yield of at least about 85%.

25. The process of claim 20, wherein the compound of formula V is compound 5:

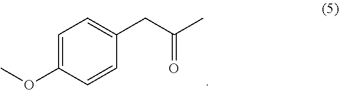

26. The process of claim 20, wherein the compound of formula VI is compound 6:

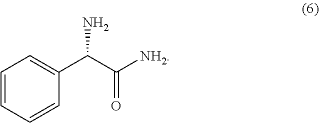 [SMILES: NC(=O)[C@@H](N)c1ccccc1]

27. The process of claim 1, further comprising:
(a) converting the compound of formula I to a salt of the compound of formula I; and
(b) converting the compound I salt to purified compound I.

28. The process of claim 27, wherein step (a) is performed using an aqueous acid, such as hydrochloric acid.

29. The process of claim 27, wherein step (a) is performed at elevated temperature, such as at least about 40° C.

30. The process of claim 27, wherein step (b) is performed by adjusting the pH to about 5 to about 6.

31. The process of claim 26, wherein step (b) further comprises crystallizing the purified D-metyrosine (compound 1).

32. The process of claim 9, further comprising
(a) heating the compound of formula III in a solvent; and
(b) cooling the solution of step (a).

33. The process of claim 32, wherein step (b) is performed at a temperature below about room temperature, such as about −20 to about 20° C., preferably about 0 to about 5° C.

34. D-metyrosine prepared according to the process of claim 1.

35. A composition comprising the D-metyrosine of claim 34.

36. The composition of claim 35, comprising a mixture of D-metyrosine and L-metyrosine,

37. The composition of claim 35, wherein the mixture comprises at least about 50 wt %, based on the weight of the composition, of D-metyrosine.

38. The composition of claim 35, wherein the mixture comprises at least about 60 wt %, at least about 70 wt %, at least about 80 wt %, at least about 90 wt %, or about 100 wt %, based on the weight of the composition, of D-metyrosine.

39. The composition of claim 35, comprising about 55 wt % of D-metyrosine and about 45 wt % of L-metyrosine, about 60 wt % of D-metyrosine and about 40 wt % of L-metyrosine, about 70 wt % of D-metyrosine and about 30 wt % of L-metyrosine, about 80 wt % of D-metyrosine and about 20 wt % of L-metyrosine, or about 90 wt % of D-metyrosine and about 10 wt %, based on the weight of the composition, of L-metyrosine.

40. The composition of claim 35, wherein the mixture comprises at least about 50 wt %, based on the weight of the composition, of L-metyrosine.

41. The composition of claim 35, wherein the mixture comprises at least about 60 wt %, at least about 70 wt %, at least about 80 wt %, at least about 90 wt %, or about 100 wt %, based on the weight of the composition, of L-metyrosine.

42. The composition of claim 35, comprising about 55 wt % of L-metyrosine and about 45 wt % of D-metyrosine, about 60 wt % of L-metyrosine and about 40 wt % of D-metyrosine, about 70 wt % of L-metyrosine and about 30 wt % of D-metyrosine, about 80 wt % of L-metyrosine and about 20 wt % of D-metyrosine, or about 90 wt % of L-metyrosine and about 10 wt %, based on the weight of the composition, of D-metyrosine.

43. A compound that is compound 2, compound 3, compound 4, compound 5, or compound 6: 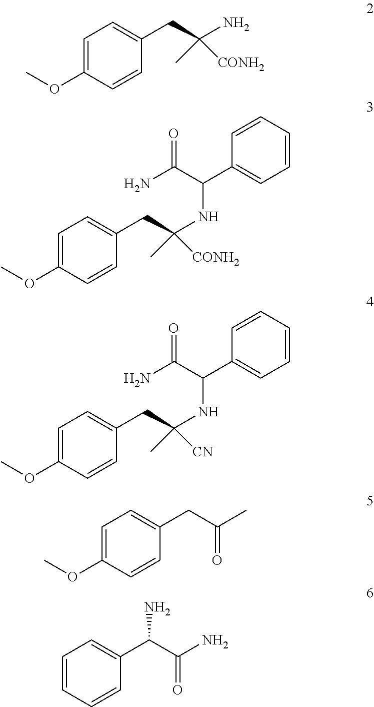
or a salt or solvate thereof.
